(19) United States
(12) Patent Application Publication
(10) Pub. No.: US 2016/0000874 A1
(43) Pub. Date: Jan. 7, 2016

(54) Modulating Ischemic Injury

(71) Applicant: Stemnion, Inc., Pittsburgh, PA (US)

(72) Inventors: Richard A. Banas, Turtle Creek, PA (US); David L. Steed, Pittsburgh, PA (US); Randall G. Rupp, Swanton, VT (US); George L. Sing, New York, NY (US)

(73) Assignee: STEMNION, INC., Pittsburgh, PA (US)

(21) Appl. No.: 14/856,000

(22) Filed: Sep. 16, 2015

Related U.S. Application Data
(63) Continuation of application No. 14/194,876, filed on Mar. 3, 2014.
(60) Provisional application No. 61/773,888, filed on Mar. 7, 2013.

(51) Int. Cl. A61K 38/19; A01N 1/02

(52) U.S. Cl. CPC A61K 38/19; A01N 1/0226

(57) ABSTRACT

The invention is directed to methods of modulating ischemic injury in tissues and organs, including donor tissue and organs and intact tissue and organs. The invention is further directed to methods of increasing time to ischemic injury in such tissues and organs. The invention is further directed to storing and preserving donor tissues and organs. Such methods utilize compositions comprising Amnion-derived Cellular Cytokine Solution (herein referred to as ACCS). The ACCS compositions may be formulated for sustained-release, targeted-release, timed-release, extended-release, etc. and may be used alone or in combination with various suitable active agents.

### FIELD OF THE INVENTION

The field of the invention is directed to methods of modulating ischemic injury in tissues and organs, including donor tissue and organs and intact tissue and organs. The field of the invention is further directed to methods of increasing time to ischemic injury in such tissues and organs. The field of the invention is further directed to storing and preserving donor tissues and organs. Such methods utilize compositions comprising Amnion-derived Cellular Cytokine Solution (herein referred to as ACCS). The ACCS compositions may be formulated for sustained-release, targeted-release, timed-release, extended-release, etc. and may be used alone or in combination with various suitable active and inactive agents.

### DESCRIPTION OF RELATED ART

PCT/US2008/00396 describes extraembryonic cells and Amnion-derived Multipotent Progenitor (AMP) cells, and/or cell lysates and/or conditioned media derived therefrom, that are useful agents capable of treating HVG, GVHD, as well as other immune and/or inflammatory diseases and disorders (incorporated herein by reference).

Banas, R. A., et al, (Human Immunology (2008) 69, 321-328) describe the immunogenicity and immunomodulatory effects of amnion-derived multipotent progenitor cells (incorporated herein by reference).

US Publication No. 20120052045 describes compositions comprising cells called Inflammatory Response Modulating Cells (IRMCs) or cell membranes derived from IRMCs. IRMCs are cells that are capable of modulating, preventing or reducing the inflammatory response and ischemic injury that occurs in a donated tissues and organs.

### BACKGROUND OF THE INVENTION

When organs are harvested for transplant, their blood supply is interrupted for various periods of time and they become ischemic organs and at normal physiologic temperatures, rapid cell death occurs (ischemic injury). In the transplant field, the standard for harvested organ preservation has been cold storage (called cold ischemic storage). Preserving the harvested organ at sub-physiologic temperature reduces cellular metabolism and slows the rate of organ cell death. The organ is generally perfused with and often immersed in solutions in an effort to further reduce damage to the cells.

Unfortunately, cold ischemic storage does not completely preserve organs and prevent ischemic injury. The three most critical problems associated with cold ischemic storage include the narrow window of time allowed for safe transport, the potential for organ damage even if the transport occurs within safe time limits, and the inability to test the organ for function after harvest, storage and transport. Although cold ischemic storage helps reduce the extent of ischemic injury, damage does occur and the more time that elapses, the more damage that occurs. And, if too much time elapses, the organ will become unusable. Even when an organ can be transplanted within the narrow time frame allotted for safe storage, the organ invariably suffers some degree of ischemic injury, which can contribute to sub-optimal outcomes in the recipient. Because transplant organs are preserved in a “non-functioning” state during cold ischemic storage they cannot be further evaluated to determine the functional status. Thus, it is virtually impossible to determine if the organ is suitable for transplant.

Clearly, a need exists for compositions and methods for preserving and storing harvested organs that can modulate, reduce or even prevent ischemic damage so that the organ remains useful and suitable for transplant. It is the object of the subject invention to provide such compositions and methods. In addition, many organs and tissues become ischemic as the result of injury, disease, surgery, etc. The methods and compositions of the invention are suitable for preventing, modulating, reducing, treating or ameliorating ischemic injury and increasing recovery from such injury in these tissues and organs, as well.

### BRIEF SUMMARY OF THE INVENTION

It is an object of the instant invention to provide novel compositions and methods for preventing, modulating, reducing, treating or ameliorating ischemic injury to tissues and organs, including donor tissue and organs and intact tissue and organs, and for storing and preserving certain tissues. It is also an object of the invention to increase the time to ischemic injury in tissues and organs, including donor tissue and organs and intact tissue and organs, and for prolonging the time a donated tissue may be preserved and stored before use. This is accomplished by perfusing, soaking or otherwise administering ACCS compositions to the tissues and organs. ACCS has been found to be capable of modulating, preventing or reducing the inflammatory response that occurs in such tissues and organs. By modulating, preventing or reducing the inflammatory response in these tissues and organs, the amount of ischemic injury in the tissue or organ is reduced. In tissues or organs destined for transplant, such treatment will cause them to be more likely to be suitable for transplant and more likely to function appropriately in the recipient. In addition, by reducing the inflammatory response and the consequent ischemic injury, tissues and organs may exhibit a longer preservation and storage time.

Accordingly, a first aspect of the invention is a method for modulating ischemic injury in tissues or organs, the method comprising the step of perfusing and/or immersing the tissue or organ with a composition comprising Amnion-derived Cellular Cytokine Solution (ACCS).

A second aspect of the invention is a method for reducing ischemic injury in tissues or organs, the method comprising the step of perfusing and/or immersing the tissue or organ with a composition comprising ACCS).

A third aspect of the invention is a method for increasing the time to ischemic injury in tissues or organs, the method comprising the step of perfusing and/or immersing the tissue or organ with a composition comprising ACCS.

A fourth aspect of the invention is a method for preserving and/or storing a tissue or organ, the method comprising the step of perfusing and/or immersing the tissue or organ with a composition comprising ACCS.

A specific embodiment of aspects 1-4 is one in which the ACCS is formulated for sustained-release, targeted-release, timed-release, or extended-release.

Another specific embodiment of aspects 1-4 is one in which the tissue or organ is a donated tissue or organ intended for transplant.

Another specific embodiment of aspects 1-4 is one in which the tissue is selected from the group consisting of epithelial tissue, connective tissue, muscle tissue and nervous tissue.

Another specific embodiment of aspects 1-4 is one in which the organ is selected from the group consisting of heart, blood vessel, alimentary canal, stomach, liver, pancreas, spleen, kidney, lung, trachea, cornea, lens, eye, bladder, ureter, urethra, uterus, ovary, testis, nerve, skin, tooth, and skeletal muscle.

Other features and advantages of the invention will be apparent from the accompanying description, examples and the claims. The contents of all references, pending patent applications and issued patents, cited throughout this application are hereby expressly incorporated by reference. In case of conflict, the present specification, including definitions, will control

### Definitions

As used herein, the terms “a” or “an” means one or more; at least one.

As defined herein “isolated” refers to material removed from its original environment and is thus altered “by the hand of man” from its natural state.

As used herein, the term “protein marker” means any protein molecule characteristic of the plasma membrane of a cell or in some cases of a specific cell type.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “Amnion-derived Multipotent Progenitor cell” or “AMP cell” means a population of epithelial cells that are derived from the amnion. AMP cells have the following characteristics. They have not been cultured in the presence of any non-human animal-derived substances or products, making them and cell products derived from them, including ACCS, suitable for human clinical use. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are isolated, will not react with antibodies to the stem/progenitor cell markers c-kit (CD117) and Thy-1 (CD90). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

As used herein, the term “substrate” means a defined coating on a surface that cells attach to, grown on, and/or migrate on. As used herein, the term “matrix” means a substance that cells grow in or on that may or may not be defined in its components. The matrix includes both biological and non-biological substances. As used herein, the term “scaffold” means a three-dimensional (3D) structure (substrate and/or matrix) that cells grow in or on. It may be composed of biological components, synthetic components or a combination of both. Further, it may be naturally constructed by cells or artificially constructed. In addition, the scaffold may contain components that have biological activity under appropriate conditions.

The term “cell product” or “cell products” as used herein refers to any and all substances made by and secreted from a cell, including but not limited to, protein factors (i.e. growth factors, differentiation factors, engraftment factors, cytokines, morphogens, proteases (i.e. to promote endogenous cell delamination, protease inhibitors), extracellular matrix components (i.e. fibronectin, etc.).

By the term “serum-free” when referring to certain compositions, growth conditions, culture media, etc. described herein, is meant that no non-human animal-derived serum is used in the preparation, growth, culturing, expansion, storage or formulation of the certain composition or process.

By the term “expanded”, in reference to cell compositions, means that the cell population constitutes a significantly higher concentration of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein. As used herein, conditioned medium also refers to components, such as proteins, that are recovered and/or purified from conditioned medium or from ECS cells, including AMP cells.

As used herein, the term “Amnion-derived Cellular Cytokine Solution” or “ACCS” means conditioned medium that has been derived from AMP cells or expanded AMP cells.

As used herein, “specific activity” means the specific activity of ACCS and compositions comprising ACCS, and is determined by calculating a 50% inhibition dosage (ID₅₀).

As used herein, the term “suspension” means a liquid containing dispersed components, i.e. cytokines The dispersed components may be fully solubilized, partially solubilized, suspended or otherwise dispersed in the liquid. Suitable liquids include, but are not limited to, water, osmotic solutions such as salt and/or sugar solutions, cell culture media, and other aqueous or non-aqueous solutions.

The term “lysate” as used herein refers to the composition obtained when cells, for example, AMP cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases.

The term “physiologic” or “physiological level” as used herein means the level that a substance in a living system is found and that is relevant to the proper functioning of a biochemical and/or biological process.

The term “therapeutically effective amount” means that amount of a therapeutic agent necessary to achieve a desired physiological effect (i.e. modulate ischemic injury).

As used herein, the term “adjunctive” means jointly, together with, in addition to, in conjunction with, and the like.

As used herein, the term “co-administer” can include simultaneous or sequential administration of two or more agents.

As used herein, the term “syngeneic” means genetically identical members of the same species.

As used herein, the term “allogeneic” means variation in alleles among members of the same species.

As used herein, the terms “immunosuppressive drugs” or “immunosuppressants” are drugs that are used in immunosuppressive therapy to inhibit or prevent activity of the immune system.

As used herein, the term “GVHD” refers to graft versus host disease, which means the processes that occur primarily in an immunocompromised host when it is recognized as non-self by immunocompetent cells of a graft.

As used herein, the term “HVG” refers to host versus graft response, which means the processes which occur when a host rejects a graft. Typically, HVG is triggered when a graft is recognized as foreign (non-self) by immunocompetent cells of the host.

As used herein, the terms “inflammation” or “inflammatory response” means the reaction that occurs in affected cells and adjacent tissues in response to an injury, insult, abnormal stimulation caused by a physical, chemical, or biologic substance, or in response to ischemic conditions.

As used herein, the term “immune response” means the cells, tissues and protein factors (i.e. cytokines) involved in recognizing and attacking foreign substances within the body of an animal.

As used herein, “ischemia” means an insufficient supply of blood to a tissue or organ.

As used herein “cold ischemic time” means the time interval that begins when a harvested tissue, organ or body part is cooled with a cold perfusion solution after organ procurement surgery and ends when the tissue or organ is implanted into the recipient.

As used herein “warm ischemic time” means the time a tissue, organ, or body part remains at physiologic body temperature after its blood supply has been interrupted but before it is cooled or reconnected to a blood supply.

As used herein, the term “pharmaceutically acceptable” means that the components, in addition to the therapeutic agent, comprising the formulation, are suitable for administration to the patient being treated in accordance with the present invention.

As used herein, the term “co-administer” can include simultaneous or sequential administration of two or more agents.

As used herein, the term “agent” means an active agent or an inactive agent. By the term “active agent” is meant an agent that is capable of having a physiological effect when administered to a subject. Non-limiting examples of active agents include growth factors, cytokines, antibiotics, cells, conditioned media from cells, etc. By the term “inactive agent” is meant an agent that does not have a physiological effect when administered. Such agents may alternatively be called “pharmaceutically acceptable excipients”. Non-limiting examples include time release capsules and the like.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion.

As used herein “subject” may mean either a human or non-human animal.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

The term “immediate-release” as used herein means that all of the pharmaceutical agent(s) is released into solution and into the biological orifice or blood or cavity etc. at the same time.

The term “targeted-release” as used herein means that the pharmaceutical agent is targeted to a specific tissue, biological orifice, tumor site or cavity, etc.

The terms “sustained-release”, “extended-release”, “time-release”, “controlled-release”, or “continuous-release” as used herein means an agent, typically a therapeutic agent or drug, that is formulated to dissolve slowly and be released over time.

As used herein the term “standard animal model” refers to any art-accepted animal model for in which the compositions of the invention exhibit efficacy.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984,“Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

The anti-inflammatory properties of Amnion-derived Cytokine Solution (ACCS) are currently being used to assist in wound healing in human clinical trials. ACCS contains more than 200 proteins, cytokines, and growth factors in solution and has been shown to reduce inflammation in several animal models (see Examples below). In addition, ACCS is presently being tested in several human clinical trials involving inflammation resulting from radiation burns and skin grafts in diabetic patients being treated for burns.

### AMP Cell Compositions

AMP cell compositions are prepared using the steps of a) recovery of the amnion from the placenta, b) dissociation of the epithelial cells from the amniotic membrane using a protease, c) culturing of the cells in a basal medium with the addition of a naturally derived or recombinantly produced human protein (i.e. human serum albumin) and no non-human animal protein; d) selecting AMP cells from the epithelial cell culture, and, optionally e) further proliferation of the cells, optionally using additional additives and/or growth factors (i.e. recombinant human EGF). Details are contained in U.S. Pat. No. 8,278,095, issued Oct. 2, 2012, U.S. Pat. No. 8,058,066, issued Nov. 15, 2011 and U.S. Pat. No. 8,088,732, issued Jan. 3, 2012, all of which are incorporated herein by reference.

Culturing of the AMP cells—The AMP cells are cultured in a basal medium. Such medium includes, but is not limited to, EPILIFE® culture medium for epithelial cells (Cascade Biologicals), OPTI-PRO™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Such media should either contain human protein or be supplemented with human protein. As used herein a “human protein” is one that is produced naturally or one that is produced using recombinant technology. “Human protein” also is meant to include a human derivative or preparation thereof, such as human serum, which contains human protein. In specific embodiments, the basal media is IMDM highly enriched basal medium, STEMLINE® T-cell expansion medium or STEMLINE® II hematopoietic stem cell expansion medium, or OPTI-PRO™ serum-free culture medium, or combinations thereof and the human protein is human serum albumin is at least 0.5% and up to 10%. In particular embodiments, the human serum albumin is from about 0.5 to about 2%. In a specific embodiment the human albumin is at 0.5%. The human albumin may come from a liquid or a dried (powder) form and includes, but is not limited to, recombinant human serum albumin, PLASBUMIN® normal human serum albumin and PLASMANATE® human blood fraction (both made by Talecris Biotherapeutics).

In a most preferred embodiment, the cells are cultured using a system that is free of non-human animal products to avoid xeno-contamination. In this embodiment, the culture medium is IMDM highly enriched basal medium, STEMLINE® T-cell expansion medium or STEMLINE® II hematopoietic stem cell expansion medium, OPTI-PRO™ serum-free culture medium, or DMEM culture medium, with human serum albumin (i.e. PLASBUMIN® normal human serum albumin) added up to amounts of 10%.

The invention further contemplates the use of any of the above basal media wherein animal-derived proteins are replaced with recombinant human proteins and animal-derived serum albumin, such as BSA, is replaced with human serum albumin. In preferred embodiments, the media is serum-free in addition to being animal-free.

Optionally, other factors are used. In one embodiment, recombinant human epidermal growth factor (hEGF) at a concentration of between 0.01-1 μg/mL is used. In a particular embodiment, the hEGF concentration is around 10-20 ng/mL. All supplements are human clinical grade.

### Generation of ACCS

Generation of ACCS—The AMP cells of the invention can be used to generate ACCS. In one embodiment, the AMP cells are isolated as described herein and 1×10⁶ cells/mL are seeded into T75 flasks containing between 5-30 mL culture medium, preferably between 10-25 mL culture medium, and most preferably about 10 mL culture medium. The cells are cultured until confluent, the medium is changed and in one embodiment the ACCS is collected 1 day post-confluence. In another embodiment the medium is changed and ACCS is collected 2 days post-confluence. In another embodiment the medium is changed and ACCS is collected 4 days post-confluence. In another embodiment the medium is changed and ACCS is collected 5 days post-confluence. In a preferred embodiment the medium is changed and ACCS is collected 3 days post-confluence. In another preferred embodiment the medium is changed and ACCS is collected 3, 4, 5, 6 or more days post-confluence. Skilled artisans will recognize that other embodiments for collecting ACCS from AMP cell cultures, such as using other tissue culture vessels, including but not limited to cell factories, flasks, hollow fibers, or suspension culture apparatus, or collecting ACCS from sub-confluent and/or actively proliferating cultures, are also contemplated by the methods of the invention. It is also contemplated by the instant invention that the ACCS be cryopreserved following collection. It is also contemplated by the invention that ACCS be lyophilized following collection. It is also contemplated by the invention that ACCS be formulated for sustained-release, timed-release, targeted-release, extended-release, etc., following collection. Skilled artisans are familiar with cryopreservation lyophilization, and sustained-release/timed-release/targeted-release/extended-release formulation methodologies.

The ACCS of the invention is characterized by assaying for physiologically relevant cytokines secreted in the physiologically relevant range of about 5-16 ng/mL for VEGF, about 3.5-4.5 ng/mL for Angiogenin, about 100-165 pg/mL for PDGF, about 2.5-2.7 ng/mL for TGFβ2, about 0.68 μg/mL for TIMP-1 and about 1.04 μg/mL for TIMP-2.

It is also contemplated by the invention that ACCS, including pooled ACCS, be concentrated prior to use. The appropriate level of concentration required will be dependent upon the intended use and therefore will need to be empirically determined.

The compositions of the invention can be prepared in a variety of ways depending on the intended use of the compositions. For example, a composition useful in practicing the invention may be a liquid comprising an agent of the invention, i.e. ACCS in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous and muco-adhesive.

### Alternative Formulation of ACCS

The ACCS may be formulated as sustained-release/controlled-release/timed-release/targeted-release, etc., compositions. Skilled artisans are familiar with methodologies to create such compositions of therapeutic agents, including protein-based therapeutic agents such as ACCS. In addition, ACCS may be formulated as a spray, gel, slave, etc.

Pharmaceutical Compositions—The present invention provides pharmaceutical compositions of ACCS and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

One of skill in the art may readily determine the appropriate concentration, or dose, of ACCS for a particular purpose. The skilled artisan will recognize that a preferred dose is one which produces a therapeutic effect, such as preventing and/or down-regulating the inflammatory response associated with ischemic injury, in a donor tissue or organ. Of course, proper doses of ACCS will require empirical determination at time of use based on several variables including but not limited to the type of donor tissue or organ and the like.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Preparation of AMP Cell Compositions

Amnion epithelial cells were dissociated from starting amniotic membrane using the dissociation agents PXXIII. The average weight range of an amnion was 18-27 g. The number of cells recovered per g of amnion was about 10-15×10⁶ for dissociation with PXXIII.

Method of obtaining selected AMP cells—Amnion epithelial cells were plated immediately upon isolation from the amnion. After ˜2-3 days in culture non-adherent cells were removed and the adherent cells were kept. This attachment to a plastic tissue culture vessel is the selection method used to obtain the desired population of AMP cells. Adherent and non-adherent AMP cells appear to have a similar cell surface marker expression profile but the adherent cells have greater viability and are the desired population of cells. Adherent AMP cells were cultured in basal medium supplemented with human serum albumin until they reached ˜120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reached ˜120,000-150,000 cells/cm², they were collected and cryopreserved. This collection time point is called p0.

### Example 2

### Generation of ACCS

The AMP cells of the invention can be used to generate ACCS, including pooled ACCS. The AMP cells were isolated as described above and about 1>10⁶ cells/mL were seeded into T75 flasks containing ˜10 mL culture medium as described above. The cells were cultured until confluent, the medium was changed and ACCS was collected ˜3 days post-confluence. Optionally, the ACCS is collected again after 3 days, and optionally again after 3 days. Skilled artisans will recognize that other embodiments for collecting ACCS from confluent cultures, such as using other tissue culture vessels, including but not limited to cell factories, flasks, hollow fibers, or suspension culture apparatus, etc. are also contemplated by the methods of the invention (see Detailed Description above). It is also contemplated by the instant invention that the ACCS be cryopreserved, lyophilized, irradiated or formulated for sustained-release, etc., following collection. It is also contemplated that ACCS be collected at different time points (see Detailed Description for details).

### Example 3

### Generation of Pooled ACCS

ACCS was obtained essentially as described above. In certain embodiments, ACCS was collected multiple times from an AMP cell culture derived from one placenta and these multiple ACCS collections were pooled together. Such pools are referred to as “SP pools” (more than one ACCS collection/one placenta). In another embodiment, AMP cell cultures were derived from several placentas, i.e. from 5 or 10 placentas. The AMP cells from each placenta were cultured and one ACCS collection from each culture was collected and then they were all pooled. These pools are termed “MP1 pools” (one ACCS collection/placenta, multiple placentas). In yet another embodiment, AMP cell cultures were derived from several placentas, i.e. from 5 or 10 placentas. The AMP cells from each placenta were cultured and more than one ACCS collection was performed from each AMP cell culture and then pooled. These pools are termed “MP2 pools” (more than one ACCS collection/placenta, multiple placentas).

### Example 4

### Inflammatory Model—Use of ACCS to Prevent Onset of Periodontal Disease in an Animal Model

Objective: The aim of this study was to evaluate the preventive role of ACCS in Porphyromonas gingivalis (P. gingivalis)-induced experimental periodontitis in rabbits

Methods: Eight New-Zealand White rabbits were distributed into 3 groups: 1. Untreated (n=2), 2. Control (unconditioned ACCS culture media) (n=3), and 3. ACCS (n=3). At baseline, all rabbits received silk ligatures bilaterally tied around mandibular second premolars under general anesthesia. The assigned test materials, ACCS or control, in volumes of 10 μL were topically applied to the ligated sites with a blunt needled-Hamilton Syringe from the time of ligature; control animals received ligature, but no treatment. Topical P. gingivalis-containing slurry (1 mL) was subsequently applied to induce the periodontal inflammation. The application of test materials and P. gingivalis continued for 6 weeks on an every-other-day schedule. At 6 weeks, following euthanasia, the mandibles were surgically harvested. Morphometric, radiographic and histologic evaluations were performed.

Results: Macroscopic evaluations including soft tissue assessments, crestal bone and infrabony measurements showed significant periodontal breakdown induced by P. gingivalis in control and no treatment groups at 6 weeks compared to historical ligature-alone groups (p=0.05, p=0.03, respectively). ACCS application significantly inhibited soft tissue inflammation and prevented both crestal bone loss and infrabony defect formation compared to untreated and control groups (p=0.01, p=0.05, respectively). Histologic assessments and histomorphometric measurements supported the clinical findings; ACCS treated animals demonstrated significantly less inflammation in soft tissue and less bone loss compared to the untreated and control groups (p=0.05).

Conclusions: Topical ACCS application prevents periodontal inflammatory changes and bone loss induced by P. gingivalis as shown both at clinical and histopathological level. ACCS has potential as a therapeutic approach for the prevention of periodontal diseases

### Example 5

### Inflammatory Model—Use of ACCS to Stop Progression of or Reverse Periodontal Disease in an Animal Model

Objective: The aim of this study was to evaluate the therapeutic actions of ACCS in the treatment of periodontitis induced by P. gingivalis.

Methods: The study was conducted using a two-phase rabbit periodontitis protocol: 1—Disease induction (6 weeks) and 2—Treatment (6 weeks). Periodontal disease was induced in 16 New-Zealand White rabbits by every-other-day application of topical P. gingivalis to ligatured mandibular premolars. At the end of Phase 1, 4 randomly selected rabbits were sacrificed to serve as the baseline disease group. For Phase 2, the remaining 12 rabbits were distributed into 3 groups (n=4), 1—Untreated, 2—Control (unconditioned ACCS culture media) and 3—ACCS treatment. At the end of Phase 2, morphometric, radiographic and histologic evaluations were performed on harvested mandibles.

Results: The baseline disease group exhibited experimental periodontitis evidenced by tissue inflammation and bone loss. At the end of Phase 2, the untreated group showed significant disease progression characterized by increased soft and hard tissue destruction (p=0.05). The tissue inflammation and bone loss was significantly reduced by topical ACCS compared to baseline disease and untreated groups (p=0.05; p=0.002, respectively). The control treatment also arrested disease progression compared to untreated group (p=0.01), but there was no improvement in periodontal health compared to baseline disease (p=0.4). Histopathological assessments revealed similar findings; ACCS stopped the progression of inflammatory process (p=0.003) and reversed bone destruction induced by P. gingivalis (p=0.008). The ACCS-treated group had minimal osteoclastic activity limited to crestal area compared to untreated and control groups, which showed a profound osteoclastogenic activity at the bone crest as well as at interproximal sites.

Conclusions: Topical application of ACCS stopped the progression of periodontal inflammation and resulted in tissue regeneration in rabbit periodontitis indicating its potential therapeutic efficacy.

### Example 6

### Inflammatory Model—Evaluate the Efficacy of Topically Applied ACCS to Inhibit Irritant 12-O-tetradecanoylphorbol-3-acetate (TPA) Skin Inflammation in Mice

Method: Topical treatment was given twice daily to the following groups: 1. TPA+topical control; 2. TPA+ACCS; 3. TPA+clobetasol 0.05 topical solution (the strongest available topical corticosteroid); 4. ACCS alone; 5. No treatment (the other untreated ear was measured). The endpoints for the study were ear thickness and ear weight at the end of the experiment. The thicker the ear and the more it weighs correlates with the degree of inflammation.

Results: Topically applied ACCS was effective at reducing the inflammation induced by TPA. The anti-inflammatory activity of topical ACCS reached the same level as clobetasol (a class 1 potent topical corticosteroid) by 3 days after beginning application.

Conclusion: ACCS has a strong anti-inflammatory effect when applied to skin.

### Example 7

### Inflammatory Model—Evaluate the Efficacy of Intralesional Injection of ACCS to Inhibit Irritant (TPA) Skin Inflammation in Mice

Method: Intralesional injection into the ear was given once daily to the following groups: 1. TPA+intralesional control; 2. TPA+intralesional ACCS; 3. TPA+intralesional kenalog (10 mg/ml) (a potent intralesional corticosteroid); 4. ACCS intralesional injection alone; 5. Saline sham injections to the normal untreated ear. The endpoints for the study were ear thickness and ear weight at the end of the experiment. The thicker the ear and the more it weighs correlates with the degree of inflammation.

Results: Intralesional injection of ACCS was effective at reducing the inflammation induced by TPA at all time points beginning on day 2 of daily injections. Intralesional kenalog (10 mg/ml) injections induced a hematoma at the site of injection, which led to some inflammation and that is why there is not a substantial difference in ear thickness when comparing TPA+kenalog with TPA+control.

Conclusions: Intralesional ACCS did reduce skin inflammation but the topically applied ACCS in Example 1 above had a more potent effect. There was no difference in ear weight using either ACCS or intralesional kenalog compared with TPA+control.

### Example 8

### Chronic Wound Healing Model—Effects of ACCS in an Animal Model of Chronic Wound Healing

An art-accepted animal model for chronic granulating wound was used to study the effects of ACCS on chronic wound healing (Hayward PG, Robson MC: Animal models of wound contraction. In Barbul A, et al: Clinical and Experimental Approaches to Dermal and Epidermal Repair: Normal and Chronic Wounds. John Wiley & Sons, New York, 1990.).

Results: ACCS was effective in not allowing proliferation of tissue bacterial bioburden. ACCS allowed accelerated healing of the granulating wound significantly faster than the non-treated infected control groups.

### Example 9

### Transplantation/Ischemia Model—Use of ACCS in Animal Models of Organ Transplantation

ACCS compositions are tested in animal models of organ transplant to evaluate their ability to prevent, modulate, reduce, treated or ameliorate ischemic injury in the harvested organ. Standard animal models of organ transplantation are found in the scientific literature as well as in “Handbook of Animal Models in Transplantation Research”, Edited by Donald V. Cramer, Luis Podesta and Leonard Makowka, published in 1993 by CRC Press (incorporated herein by reference).

### Example 10

### Ischemia Model—Use of ACCS on Tissue Ischemia-Reperfusion Injury

ACCS compositions are tested for their ability to prevent, modulate, reduce, treated or ameliorate ischemic tissue injuries. The experimental approach includes testing ACCS compositions in a hind limb ischemia model. This model utilizes a controlled tension tourniquet circumferentially around the proximal thigh of a mouse for 3 hours. Reperfusion is initiated by release of tension on the tourniquet. Immediately following reperfusion, ACCS compositions are injected into the left ventricle. Perfusion-restoration of blood flow is monitored by laser Doppler flow imaging. Immunohistochemistry and quantitative PCR are used to assess accelerated tissue neovascularization and angiogenesis.

### Example 11

### Preservation/Storage—Use of ACCS as a Storage Preservative for Donated Tissues and Organs

The successful outcome of the majority of corneal transplants depends on the presence of a viable corneal endothelium. Since human corneal endothelial cells do not readily proliferate, preservation of the endothelium is a primary aim of methods of corneal storage. Although some cryopreserved corneas have been transplanted successfully, the complexity of the standard cryopreservation technique and its potential for causing endothelial damage have limited its application. Because of its anti-inflammatory properties, ACCS is tested for its ability to preserve corneas for transplant.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

1.-20. (canceled)

21. A method for modulating ischemic injury in an ischemic lung, the method comprising the step of administrating a composition comprising Amnion-derived Cellular Cytokine Solution (ACCS) to the ischemic lung such that the ischemic injury is modulated, wherein the ACCS is formulated for spray administration.

22. The method of claim 21 wherein the ACCS is further formulated for sustained-release, targeted-release, timed-release, or extended-release.

23. A method for reducing ischemic injury in an ischemic lung, the method comprising the step of administrating a composition comprising ACCS to the ischemic lung such that the ischemic injury is reduced, wherein the ACCS is formulated for spray administration.

24. The method of claim 23 wherein the ACCS is further formulated for sustained-release, targeted-release, timed-release, or extended-release.

25. A method for increasing the time to ischemic injury in a lung at risk for developing ischemic injury, the method comprising the step of administrating a composition comprising ACCS to the ischemic lung such that the time to ischemic injury is increased, wherein the ACCS is formulated for spray administration.

26. The method of claim 25 wherein the ACCS is further formulated for sustained-release, targeted-release, timed-release, or extended-release.
